(19) United States
(12) Patent Application Publication
(10) Pub. No.: US 2004/0038904 A1
(43) Pub. Date: Feb. 26, 2004

(54) Method of treating multiple sclerosis

(76) Inventor: Angela Ogden, Long Valley, NJ (US)

Correspondence Address: Pharmacia & Upjohn Company, Global Intellectual Property, 301 Henrietta Street, Kalamazoo, MI, 49001, US

(21) Appl. No.: 10/438,131

(22) Filed: May 14, 2003

Related U.S. Application Data
(60) Provisional application No. 60/382,159, filed on May 21, 2002.

(51) Int. Cl. A61K 38/08; A61K 31/704

(52) U.S. Cl. 514/16; 514/34

(57) ABSTRACT

The present invention provides for use of an anthracycline, such as doxorubicin, alone or in combination with a protective agent, such as dexrazoxane, for treating multiple sclerosis.

### CROSS-REFERENCE TO RELATED APPLICATION

This application claims the benefit of U.S. provisional application Serial No. 60/382,159, filed May 21, 2002, under 35 U.S.C. §119 (e)(1).

### BACKGROUND OF THE INVENTION

1. Field of the Invention

The present invention relates to treatment of multiple sclerosis, and more specifically to the use of anthracyclines, alone or in combination with a protective agent, to treat multiple sclerosis.

2. Description of the Related Art

Multiple Sclerosis (MS) is a disease of the central nervous system that affects the brain and spinal cord. It strikes an estimated 250,000 people in the United States and is the major acquired neurologic disease in young adults. Common signs and symptoms of MS include fatigue, psychological and cognitive changes, weakness or paralysis of limbs, numbness, vision problems, speech difficulties, muscle spasticity, difficulty with balance when walking or standing, bowel and bladder dysfunction, and sexual dysfunction. Approximately half the people with this disease have relapsing-remitting MS in which there are unpredictable attacks where the clinical symptoms become worse (exacerbation) which are separated by periods of remission where the symptoms stabilize or diminish. The other half have chronic progressive MS without periods of remission.

At present there are no cures for MS. Many medications are available to relieve symptoms in progressive MS. For example, corticosteroids are used to reduce inflammation in nerve tissue and shorten the duration of flare-ups; Muscle relaxants such as tizanidine (Zanaflex) and baclofen (Lioresal) are oral treatments for muscle spasticity; Antidepressant medication fluoxetine (Prozac), the antiviral drug amantadine (Symmetrel) or a medication for narcolespy called modafinil (Provigil) are used to reduce fatigue.

A few other drugs are available for MS that are not directly related to symptom management and but may act to alter the course of the disease. These drugs include beta interferons (Betaferon, Avonex, Rebif) and glatiramer acetate (Copaxone). These drugs may have an impact on the frequency and severity of relapses, and the number of lesions as seen on MRI scans. Some of the drugs appear to have an effect of slowing the progression of disability. U.S. Pat. No. 4,617,319 discloses a method of treating multiple sclerosis using 1,4-dihydroxy-5,8-bis[[(2-hydroxyethylamino)ethyl]amino]anthraquinone, which is also known by the generic name mitoxantrone. Mitoxantrone is a synthetic anthracenedione and is the active ingredient of the antineoplastic drug Novantrone®.

None of these existing therapies are proven satisfactory because of limited efficacy and /or significant toxicity. In addition, many of these therapies are required to be administered frequently and some are very expensive. Thus, there clearly exists a need for novel and effective methods of treating MS.

Anthracyclines are members of a very important class of antineoplastic agents that has been used clinically for decades in a wide range of human tumors. Examples of commonly used anthracyclines include doxorubicin, daunorubicin, epirubicin, and idarubicin. This class of agents also possesses antibacterial activities.

Doxorubicin is effective as an anti-tumor agent against a variety of neoplasms such as acute leukemias and malignant lymphomas. It is also very effective in the treatment of solid tumors, particularly when administered as part of a combination regimen. Doxorubicin is commercially available under the trade names Adriamycin RDF®/PFS® (doxorubicin hydrochloride injection, USP) from Pharmacia & Upjohn, Doxil® (doxorubicin HCl liposome injection) from Alza, Lipodox® from Pfizer, DaunoXome® from Nexter, MTC doxo (doxorubicin magnetic targeted particles) from FeRx/Elan, and Rubex® (doxorubicin hydrochloride for injection) from Bristol-Myers Squibb Oncology/Immunology. Chemically, doxorubicin hydrochloride is (8 S,10 S)-10-[(3-amino-2,3,6-trideoxy-(alpha)-L-lyxo-hexopyranosyl)oxy]-8-glycolyl-7,8,9,10-tetrahydro-6,8,11-trihydroxy-1-methoxy-5,12-naphthacenedione hydrochloride.

Epirubicin is used to treat some kinds of cancers of the breast, lung, lymph system, stomach, and ovaries. Epirubicin hydrochloride is commercially available under the trade name Ellence® (Pharmacia & Upjohn). Chemically, epirubicin hydrochloride is (8S-cis)-10-[(3-amino-2,3,6-trideoxy-(alpha)-L-arabino-hexopyranosyl)oxy]-7,8,9,10-tetrahydro-6,8,11-trihydroxy-8-(hydroxyacetyl)-1-methoxy-5,12-naphthacenedione hydrochloride.

Daunorubicin is used to treat acute nonlymphocytic leukemia (myelogenous, monocytic, erythroid) of adults and in acute lymphocytic leukemia of children and adults. Daunorubicin hydrochloride is commercially available under the trade name Cerubidine from Bedford. Chemically, daunorubicin hydrochloride is (1 S ,3 S )-3-Acetyl-1,2,3,4,6,11-hexahydro-3,5,12-trihydroxy-10-methoxy-6,11-dioxo-1-naphthacenyl 3-amino-2,3,6-trideoxy-(alpha)-L-lyxo-hexopyranoside hydrochloride.

Examples of other anthracyclines or of anthracycline derivatives developed or explored for use as antineoplaxtic agents include 4′ deoxy-4′-iododoxorubicin (U.S. Pat. No. 4,438,105), nemorubicin (U.S. Pat. No. 4,672,057), AR522 (liposome annamycin, Aronex, CLIN. CANC. RES. Jan. 11, 1995 (1369-1374), L 377202 (Chemical Name: (4R)-1-(4-carboxy-1-oxobutyl)-4-hydroxy-L-prolyl-L-alanyl-L-seryl-(2R)-2-cyclohexylglycyl-L-glutaminyl-L-seryl-L-leucine), Merck & Co), and GPX-100 (anthracycline, Gem Pharm).

Despite the effectiveness of anthracyclines as clinical antineoplastic agents, it is known that, like many other antineoplastic agents, anthracyclines have serious side effects such as cardiotoxicity, bone-marrow depression and gastrointestinal tract mucositis, which significantly limit their clinical usefulness.

U.S. Pat. No. 6,057,361 discloses a method of reducing anthracycline toxicity by administration of dimesna and analogues and derivatives thereof.

U.S. Pat. No. 6,147,094 361 discloses a method of reducing anthracycline-induced cardiotoxicity by administration of manganese compounds.

U.S. Pat. No. 5,242,901 discloses a method of reducing anthracycline-induced cardiotoxicity by administration of a protective agent such as dexrazoxane.

U.S. Pat. No. 5,744,455 discloses a human anti-neoplastic composition comprising an anthracycline in admixture with dexrazoxane.

U.S. Pat. No. 4,257,063 discloses a pharmaceutical composition useful for aiding regression and palliation of sarcoma, lymphosarcoma, and leukaemia in humans which comprises an amount therapeutically effective in aiding said regression and palliation of dexrazoxane.

Franz X, et al. disclose an experimental study on the effect of mitoxantrone in combination with dexrazoxane on experimental autoimmune encephalomyelitis in Lewis Rats. (Franz X et al. Combination therapy with the cardioprotector dexrazoxane augments therapeutic efficacy of mitoxantrone in experimental autoimmune encephalomyclitis in Lewis Rats. Neurology 54 (Supplement 3): A60-61 (2000)) Dexrazoxane is currently marketed under the trade name Zinecard™ by Pharmacia, Inc. as a cardioprotective agent. Chemically, dexrazoxane is (S)-4,4′-(1-methyl-1,2-ethanediyl)bis-2,6-piperazinedione.

Surprisingly and unexpectedly, it has been found that anthracyclines can be used to treat MS, either alone or in combination with administration of protective agent.

### SUMMARY OF INVENTION

It is an object of the invention to provide a novel method of treating multiple sclerosis.

It is another object of the invention to provide a method of treating multiple sclerosis wherein the toxic effects of the active therapeutic agent are reduced or minimized.

It is yet another object of the invention to provide a method of treating multiple sclerosis that is convenient for the patient.

It is yet another object of the invention to provide a novel use of anthracyclines.

It is still another object of the invention to provide a composition comprising an anthracycline for use as treatment of multiple sclerosis.

These and other objects are met by the present invention. In one aspect, the invention provides for a method of treating MS in a patient suffering from MS and in need of treatment comprising the administration of a therapeutically effective amount of one or more anthracyclines or pharmaceutically acceptable salts thereof. Specific anthracyclines suitable for the present invention includes doxorubicin, daunorubicin, epirubicin, idarubicin, doxorubicin, daunorubicin, epirubicin, idarubicin, menogaril, aclarubicin, zorubicin, pirarubicin, valrubicin, amrubicin, and pharmacologically acceptable salts thereof.

The anthracyclines are administered at relatively long intervals, generally every 7 to 15 weeks, thus making the treatment more convenient for the patients.

In another aspect, the invention provides for a method of treating MS in a patient suffering from MS and in need of treatment comprising the administration of a therapeutically effective amount of one or more anthracyclines in combination with administration of an effective amount of a protective agent. One example of the protective agent is bisdioxopiperazine. Another example of the protective agent is a is a compound of formula (I):

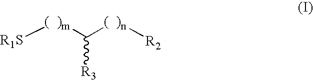

or a pharmaceutically acceptable salt thereof, wherein in formula (I):

R₁ is hydrogen, lower alkyl or

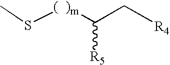

R₂ and R₄ are each individually SO₃⁻M⁺, PO₃²⁻M₂²⁺, or PO₂S²⁻M₂²⁺;

R₃ and R₅ are each individually hydrogen, hydroxy or sulfhydryl;

m and n are individually 0, 1, 2, 3 or 4, with the proviso that if m or n is 0, then R₃ is hydrogen; and

M is hydrogen or an alkali metal ion; or

Still another example of the protective agent is a compound of formula (II):

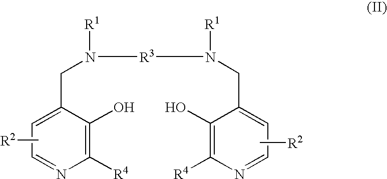

or a metal chelate thereof or salt of a metal chelate thereof, wherein in formula (II),

each R¹ independently represents hydrogen or —CH₂ COR⁵;

R⁵ represents hydroxy, optionally hydroxylated alkoxy, amino or alkylamido;

each R² independently represents a group XYR⁶;

X represents a bond, or a C₁₋₃ alkylene or oxoalkylene group optionally substituted by a group R⁷;

Y represents a bond, an oxygen atom or a group NR⁶;

R⁶ is a hydrogen atom, a group COOR⁸, an alkyl, alkenyl, cycloalkyl, aryl or aralkyl group optionally substituted by one or more groups selected from COOR.sup.8, CONR⁸₂, NR⁸₂, OR⁸, ═NR⁸, ═O, OP(O)(OR⁸)R⁷ and OSO₃ M;

R⁷ is hydroxy, an optionally hydroxylated, optionally alkoxylated alkyl or aminoalkyl group;

R⁸ is a hydrogen atom or an optionally hydroxylated, optionally alkoxylated alkyl group;

M is a hydrogen atom or one equivalent of a physiologically tolerable cation;

R³ represents a C₁₋₈ alkylene group, a 1,2-cycloalkylene group, or a 1,2-arylene group; and

each R⁴ independently represents hydrogen or C₁₋₃ alkyl.

The administration of the protective agent reduces the toxic effects of the anthracyclines, which not only makes the treatment more tolerable to the patients, but also permits higher doses of anthracyclines to be administered or permits the patients to be on the therapy for a longer period of time.

### DETAILED DESCRIPTION OF THE INVENTION

In one aspect, the invention provides for a method of treating MS in a patient suffering from MS and in need of treatment comprising administering to the patient a therapeutically effective amount of one or more anthracyclines or pharmaceutically acceptable salts thereof.

The term “treat,” “treating,” or “treatment” as used herein refers to ameliorating or alleviating one or more symptoms of MS or altering the course of the disease, or both, in a patient to which an anthracycline is administered.

The term “pharmaceutically acceptable” as used herein refers to those properties and/or substances which are acceptable to the patient from a pharmaco-logical/toxicological point of view and to the manufacturing pharmaceutical chemist from a physical/chemical point of view regarding composition, formulation, stability, patient acceptance and bioavailability.

The term “anthracycline” as used herein refers to a compound of the anthracycline class of natural products and the synthetic or semi-synthetic analogs or derivatives thereof Examples of the natural products of the anthracycline class are daunorubicin and doxorubicin, which are produced by microorganisms belonging to the genus Streptomyces. These compounds can be structurally defined as glycosides whose aglycone is characterized by a tetracyclic anthraquinone chromophore. Members of the anthracycline class are useful as antineoplastic agents.

Any anthracyclines, including both natural and derivatives, particularly those that are used or suitable for clinical use as antienoplastic agents in cancer chemotherapy, can be used in the present invention. Examples of anthracyclines suitable for the invention, and the synthesis thereof, are described in A. Suarato, F. Angelucci, and A Bargiotti: Antitumor Anthracyclines, Chimicaoggi, 9-19 (April 1990); J W Lown: Anthracycline and Anthraquinone Anticancer Agents: Current Status and Recent Developments. Pharmac. Ther. 60:185-214 (1993); F M Arcamone: From the Pigments of the Actinomycetes to Third Generation Antitumor Anthracyclines, Biochimie, 80, 201-206 (1998); C Monneret: Recent Development in the Field of Antitumour Anthracyclines, Eur. J. Med. Chem. 36: 483-493 (2001); and U.S. Pat. Nos. 4,438,015, 4,672,057, 5,646,177, 5,801,257, and 6,284,737. The disclosure of the above references is incorporated herein by reference. Examples of particular anthracyclines suitable for the invention include, but not limited to, doxorubicin, 13-deoxydoxorubicin (also known as GPX-100), iodoxorubicin, daunorubicin, epirubicin, THP-adriamycin, idarubicin, menogaril, aclacinomycin A (also known as aclarubicin), zorubicin, pirarubicin, valrubicin, amrubicin, iodoxorubicin, nemorubicin, (4R)-1-(4-carboxy-1-oxobutyl)-4-hydroxy-L-prolyl-L-alanyl-L-seryl-(2R)-2-cyclohexylglycyl-L-glutaminyl-L-seryl-L-leucine (also known as L 377202), 4′ deoxy-4′-iododoxorubicin, and salts thereof.

The anthracyclines of the present invention can be administered as primary drugs in their active forms, or administered as anthraycycline prodrugs. The term “anthracycline prodrug” as used herein refers to a compound that can be converted to a biologically active anthracycline, either in vivo after administration or in vitro prior to administration of the compound. A prodrug may have no or minimal therapeutic activity until it is converted to its biologically active form. An anthracycline prodrug can be a compound that contains an anthracycline having one or more functional groups covalently bound to a blocking moiety. Examples of anthracycline prodrugs suitable in the present invention, and the synthesis thereof, are described by, for example, Leenders, et al. in U.S. Pat. No. 5,710,135, by Barbas, III, et al. in U.S. Pat. No. 6,268,488, by J. lacquesy et al. WO 92/19639, by K. Bosslet et al. Cancer Res. 54: 2151-2159 (1994), by S. Andrianomenjanahary et al. Bioorg. Med. Chem Lett. 2:1093-1096 (1992) and by J. -P. Gesson et al. Anti-Cancer Drug Des. 9: 409-423 (1994).

The term “therapeutically effective amount” of an anthracycline as used herein refers to any amount of the anthracycline that is sufficient to treat MS in a patient. When the anthracyclines are administered in prodrug forms, the “therapeutically effective amount” refers to the amount of the active anthracycline that is converted from the anthracycline prodrug. The specific therapeutically effective amount will vary with such factors as the particular anthracycline used, specific formulations employed, mode and route of administration, the physical condition of the patient, duration of the treatment, and nature of concurrent therapy (if any). The dosage of an anthracycline in the present invention can be from about 1 mg to 1000 mg/m² or higher, but is generally the same or less than the dosage normally used in, or suitable for, cancer chemotherapy for that anthracycline. Due to potential toxic effects of anthracyclines, patients treated with anthracyclines should be periodically monitored during the course of therapy for potential hematologic toxicity, such as bone marrow depression, and non-hematologic toxicity, such as cardiomyopathy. The severity of the hematologic and non-hematologic toxicity can be assessed by methods known in the art, such as using the National Cancer Institute Common Toxicology Criteria (also known as “NCI-CTC”). NCI-CTC is available online at http://ctep.cancer.gov/reporting/ctc.html. Generally, the treatment is initiated with lower doses and, if the hematologic and non- hematologic toxicity does not exceed grade 22 by the NCI-CTC criteria, the doses may be escalated gradually in the next cycle until an optimal dose is reached. On the other hand, if sustained hematologic toxicity occurs, reduction or suspension or delay of anthracycline therapy should be considered. If deterioration in cardiac function of the patient occurs, anthracycline therapy may be discontinued.

Anthracyclines of the present invention can be administered in cycles over 7-week to 15-week intervals. Generally, treatment with anthracyclines is started with a 12-week cycle and the patient is monitored for progress of the treatment during the course of treatment. If the condition of the patient deteriorates between week 8 and 12 of the cycle, the treatment cycle should be shortened to, for example, 9 weeks or shorter.

The preferred mode for administering the anthracyclines is parenteral, e.g. intravenous administration and the total dose of the anthracycline for each cycle can be injected slowly into the patient in a single dose or in divided doses administered within a day. The rate of intravenous administration is dependent on such factors as the size of the vein, the specific anthracycline, dosage, characteristics of the formulation, condition of the patient, and generally is not less than 3 to 5 minutes.

Anthracyclines of the present invention may be formulated with conventional pharmaceutical formulation aids, for example stabilizers, antioxidants, osmolality adjusting agents, buffers, pH adjusting agents, etc. and may be in a conventional pharmaceutical administration form such as a tablet, capsule, powder, solution, suspension, dispersion, syrup, suppository, etc. However, solutions, suspensions and dispersions in physiologically acceptable carrier media, for example water for injections, is generally preferred.

Parenterally administrable forms, e.g. intravenous solutions, suspension, or dispersions, should be sterile and should have low osmolality to minimize irritation or other adverse effects upon administration, and thus the compositions should preferably be isotonic. Suitable vehicles include aqueous vehicles customarily used for administering parenteral dosage forms such as Sodium Chloride Injection, Ringer's Injection, Dextrose Injection, Dextrose and Sodium Chloride Injection, Lactated Ringer's Injection and other solutions such as are described in Remington's Pharmaceutical Sciences, 15th ed., Easton: Mack Publishing Co., pp. 1405-1412 and 1461-1487 (1975) and The National Formulary XIV, 14th ed. Washington: American Pharmaceutical Association (1975). The solutions may contain preservatives, antimicrobial agents, buffers and antioxidants conventionally used for parenteral solutions, excipients and other additives which are compatible with the anthracyclines and which will not interfere with the manufacture, storage or use of the products. The liquid dosage forms for parenteral administration, will generally contain the anthracyclines at a concentration in the range of from 0.1 to 5.0 mg/mL, preferably 0.5 to 3 mg/mL. If convenient, the therapeutic agent may be supplied in a more concentrated form for dilution prior to administration.

Information on the dosages, dosage forms, frequency and route of administration of exemplary anthracyclines in the present invention is provided below. The pharmaceutical compositions and dosage forms of these anthracyclines currently available on the market can conveniently and preferably be used in the present invention. Description of the commercial pharmaceutical compositions and dosage forms of doxorubicin, daunorubicin, epirubicin, idarubicin, and other anthracyclines that are available on the market can be readily found in the product inserts or in the Physician Desk Reference. The compositions, dosage forms, and dosing regimen for anthracyclines, e.g., doxorubicin, daunorubicin, epirubicin, and idarubicin, for treating MS in the present invention set forth below apply whether or not dexazoxane is administered to the patient to which the anthracycline is administered.

Currently, doxorubicin hydrochloride is available under the various trade names, for example, Adriamycin RDF®/PFS®, Doxil®, Lipodox®, Caelyx®, DanunoXome®, and Rubex®. Adriamycin RDF® is a sterile lyophilized powder for intravenous use and is available in 10, 20 and 50 mg single dose vials and a 150 mg multidose vial. Each 10 mg single dose vial contains 10 mg of doxorubicin HCl, USP, 50 mg of lactose, NF (hydrous) and 1 mg of methylparaben, NF (added to enhance dissolution) as a sterile lyophilized powder. Each 20 mg single dose vial contains 20 mg of doxorubicin HCl, USP, 100 mg of lactose, NF (hydrous) and 2 mg of methylparaben, NF (added to enhance dissolution) as a sterile lyophilized powder. Each 50 mg single dose vial contains 50 mg of doxorubicin HCl, USP, 250 mg of lactose, NF (hydrous) and 5 mg of methylparaben, NF (added to enhance dissolution) as a sterile red-orange lyophilized powder. Each 150 mg multidose vial contains 150 mg of doxorubicin HCl, USP, 750 mg of lactose, NF (hydrous) and 15 mg of methylparaben, NF (added to enhance dissolution) as a sterile lyophilized powder.

Rubex® is also provided as lyophilized powder in 50 mg and 100 mg vials. The 50 mg and 100 mg vials is reconstituted with 25 mL and 50 mL, respectively, of a pharmaceutically acceptable diluent, such as Sodium Chloride Injection, USP (0.9%), to give a final concentration of 2 mg/mL of doxorubicin hydrochloride.

Adriamycin PFS® (doxorubicin hydrochloride injection, USP) is a sterile parenteral, isotonic solution for intravenous use, available in 5 mL (10 mg), 10 mL (20 mg), 25 mL (50 mg), and 37.5 mL (75 mg) single dose vials and a 100 mL (200 mg) multidose vial. Each mL contains doxorubicin HCl 2 mg, USP and the following inactive ingredients: sodium chloride 0.9% and water for injection q.s. Hydrochloric acid is used to adjust the pH to a target pH of 3.0.

Doxil® is doxorubicin hydrochloride (HCl) encapsulated in Stealth® liposomes for intravenous administration. Doxil® is provided as a sterile liposomal dispersion in 10-mL or 30-mL glass vials. Each vial contains 20 mg or 50 mg doxorubicin HCl at a concentration of 2 mg/mL and a pH of 6.5. The STEALTH® liposome carriers are composed of N-(carbonyl-methoxypolyethylene glycol 2000)-1,2-distearoyl-sn-glycero-3-phosphoethanolamine sodium salt (MPEG-DSPE), 3.19 mg/mL; fully hydrogenated soy phosphatidylcholine (HSPC), 9.58 mg/mL; and cholesterol, 3.19 mg/mL. Each mL also contains ammonium sulfate, approximately 2 mg; histidine as a buffer; hydrochloric acid and/or sodium hydroxide for pH control; and sucrose to maintain isotonicity. Greater than 90% of the drug is encapsulated in the STEALTH® liposomes. Other liposomal formulations for doxorubicin HCl include Lipidox® or TLC D-99 developed by Pfizer, DanunoXome® from Nexstar.

Generally, the dose schedule for doxorubicin when used as a single intravenous injection is from about 10 mg/m² to about 60 mg/m²administered at 7-week to 15-week intervals, typically from about 35 mg/m² to about 45 mg/m² administered at 8 week to 12 week intervals. The lower dosage should be given to patients with inadequate marrow reserves due to old age, prior therapy, or other conditions. Doxorubicin dosage should be reduced in case of hyperbilirubinemia.

In another embodiment, the present invention is directed to a method of treating MS comprising the administration of an effective amount of epirubicin, a derivative thereof, or a pharmaceutically acceptable acid addition salt. An example of the pharmaceutically acceptable acid addition salt is epirubicin hydrochloride. It is preferred that epirubicin is administered intravenously. Formulations suitable in the present invention can be prepared by methods known in the art. Examples of formulations suitable for intravenous administration are the commercial products for epirubicin hydrochloride under the trade name Ellence. The dose of epirubicin by single intravenous injection is generally from about 30 to about 150 mg/m² in 7-week to 12-week intervals, and is typically from 75 to about 100 mg/m² in 8-week to 12-week intervals.

In still another embodiment, the present invention is directed to a method of treating MS comprising the administration of an effective amount of daunorubicin, a derivative thereof, or a pharmaceutically acceptable acid addition salt. An example of the pharmaceutically acceptable acid addition salt is daunorubicin hydrochloride. It is preferred that daunorubicin is administered intravenously. Formulations suitable in the present invention can be prepared by methods known in the art. An examples of formulations suitable for intravenous administration is a commercial product for daunorubicin hydrochloride under the trade name Cerubidine. Cerubidine (daunorubicin HCl) for Injection, is available in butyl-rubber-stoppered vials, each containing 21.4 mg daunorubicin hydrochloride equivalent to 20 mg of daunorubicin and 100 mg of mannitol, as a sterile lyophilized powder. The lyophilized powder should be reconstituted with a pharmaceutically acceptable diluent such as Sterile Water for Injection, USP, before administration.

The dose of daunorubicin by single intravenous injection is generally from about 30 to about 100 mg/m² administered in 7-week to 12-week cycles, and typically from 40 to about 60 mg/m² in 8-week to 12-week cycles. The dose should be reduced in instances of hepatic or renal impairment.

In yet another embodiment, the present invention is directed to a method of treating MS comprising the administration of an effective amount of idarubicin, a derivative thereof, or a pharmaceutically acceptable acid addition salt, with idarubicin hydrochloride being preferred. It is preferred that idarubicin is administered intravenously. Formulations suitable in the present invention can be prepared by methods known in the art. An example of formulations suitable for intravenous administration in the present invention is a commercial product for idarubicin hydrochloride under the trade name Idamycin PFS. Idamycin PFS is a sterile, isotonic parenteral preservative-free solution, available in 5 mL (5 mg), 10 mL (10 mg) and 20 mL (20 mg) single use only vials. Each mL contains Idarubicin HCl, USP 1 mg and the following inactive ingredients: Glycerin, USP 25 mg and Water for Injection, USP q.s. Hydrochloric Acid, NF is used to adjust the pH to a target of 3.5.

The dose of idarubicin as a single dose by intravenous administration is generally from about 12 to about 60 mg/m² in repeated 7-week to 12-week cycles, and typically from about 40 to about 60 mg/m² in repeated 8-week to 12-week cycles. The dose of reduction of idarubicin in patients with hepatic and/or renal impairment should be considered. Generally, administration of idarubicin should stop if the bilirubin level exceeds 5 mg %.

In another aspect, the invention provides for a method of treating MS in a patient suffering from MS and in need of treatment comprising administering to the patient a therapeutically effective amount of one or more anthracyclines in combination with an effective amount of a protective agent. The term “effective amount” of a protective agent as used herein refers to any amount of the protective agent that is sufficient to reduce the severity or extent of toxic side effects that may be caused by the anthracycline-type compound in a patient. The term “protective agent” as used herein refers to any compound that is suitable for administering to humans and is capable of reducing the toxic effects of the anthracyclines administered. In one aspect, the protective agent in the present invention is a bisdioxopiperazine. It is preferred that the bisdioxopiperazine is (+)-1,2-bis(3,5-dioxopiperazinyl-1-yl)propane, which is also known as is (S)-4,4′-(1-methyl-1,2-ethanediyl)bis-2,6-piperazinedione and ICRF-187, and generically known as dexrazoxane.

Bisdioxopiperazine can be prepared by the procedure described in U.S. Pat. No. 3,941,790. Formulations suitable in the present invention can be prepared by methods known in the art. U.S. Pat. No. 4,275,063 describes a pharmaceutical composition useful for aiding regression and palliation of sarcoma, lymphosarcoma and leukemia in animals containing these compounds as the active agent. An example of formulations suitable for intravenous administration in the present invention is a commercial product for dexrazoxane under the trade name Zinecard® (dexrazoxane for injection). Zinecard® is a sterile, pyrogen-free lyophilizate intended for intravenous administration. Zinecard® is available in 250 mg and 500 mg single use only vials. Each 250 mg vial contains dexrazoxane hydrochloride equivalent to 250 mg dexrazoxane. Hydrochloric Acid, NF is added for pH adjustment. When reconstituted as directed with the 25 mL vial of 0.167 Molar (M/6) Sodium Lactate Injection, USP diluent provided, each mL contains 10 mg dexrazoxane. The pH of the resultant solution is 3.5 to 5.5. Each 500 mg vial contains dexrazoxane hydrochloride equivalent to 500 mg dexrazoxane. Hydrochloric Acid, NF is added for pH adjustment. When reconstituted as directed with the 50 mL vial of 0.167 Molar (M/6) Sodium Lactate Injection, USP diluent provided, each mL contains 10 mg dexrazoxane. The pH of the resultant solution is 3.5 to 5.5.

Dexrazoxane can be administered by single intravenous infusion or injection at doses of between 100 and 2500 mg/m². The doses of dexrazoxane should be adjusted in accordance with several factors such as the potency of the anthracycline in causing toxic effect and the doses of the anthracycline being administered. Generally the dose of dexrazoxane is approximately 10 times the dose of doxorubicin or epirubicin dose administered, and 20 times the dose of daunorubicin or idarubicin administered. The dose frequency for dexrazoxane generally is the same as that for the anthracycline used as set forth above.

Dexrazoxane can be administered between about one hour prior to the administration of the anthracycline to about one hour after the administration of the anthracycline. Preferably, dexrazoxane is administered within about 30 to 45 minutes before, or simultaneously with, the administration of the anthracycline-type compound. Most preferably, dexrazoxane is administered about 30 minutes before administration of the anthracycline-type compound. Other schedules for the relative administration of dexrazoxane and the anthracycline can be readily determined based on the above discussion, by routine experimentation.

In one aspect, the protective agent in the present invention is a compound of formula (I):

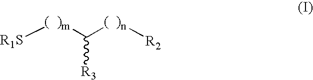

or a pharmaceutically acceptable salt thereof, wherein in formula (I),

R₁ is hydrogen, lower alkyl or

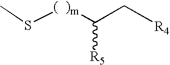

R₂ and R₄ are each individually SO₃⁻M⁺, PO₃²⁻M₂²⁺, or PO₂S^{2<}M₂²⁺;

R₃ and R₅ are each individually hydrogen, hydroxy or sulfhydryl;

m and n are individually 0, 1, 2, 3 or 4, with the proviso that if m or n is 0, then R₃ is hydrogen; and

M is hydrogen or an alkali metal ion.

Particular compounds in formula (I) useful in the present invention include Dimesna (Disodium-2,2′-dithiobis ethane sulfonate), the disphosphonate analogue of Dimesna (dimephos), the heterodimer of Mesna, where R2 is sulfonate, R4 is phosphonate (mesnaphos), S-methyl Mesna, and those analogues where one or both of R3 and R5 are hydroxy and m and n are at least 1 (hydroxymesna).

Compounds of formula (I), their preparations, formulations, and administration are disclosed in U.S. Pat. No. 6,057,361, the disclosure of which is incorporated herein be reference, and are briefly provided herein below.

Compounds of formula (I) can be administered by any suitable routes, such as by oral and parenteral administration. It is usually preferred that compounds of formula (I) are administered parenterally. To ensure maximum effect, the formula (I) compound should be administered such that a suitable concentration of the formula (I) compound is present in the body to react with the anthracycline and/or metabolites thereof. Preferred timing of the dosage of the formula (I) compound will depend upon the pharmacologic properties of the particular anthracycline, generally from about one minute prior to the administration of the anthracycline to about one hour prior to such administration. A preferred initial route of administration of the formula (I) compound at this time is by a single IV push, which is administered between fifteen and thirty minutes prior to the start of administration of the anthracycline.

The doses of the compounds of formula (I) varies depending on many factors such as the specific formula (I) compound used and the doses and formulations of the specific anthracycline used. Generally, the dose ratio, by dose weight, of the anthracycline to the formula (I) compound ranges from 1:5 to 1:4000. These ratios are applicable for all routes of initial administration of the formula (I) compound and the anthracycline, whether the two are administered simultaneously or staggered, and whether the two are administered in the same or separate formulations.

The formula (I) compounds may be formulated in combination with the anthracycline in a single formulation, or formulated apart from the anthracycline. The concentration of the Formula (I) compound in any given parenteral formulation is determined by the final desired form. If the final form is a solution, the upper limit of the concentration of the Formula (I) compound is its maximum solubility in the solvent or solvents selected. If the final form is a suspension, the concentration may be higher. For oral dosage forms, the total amount of Formula (I) compound present in the dose is preferably an amount which will allow a recommended dose to be conveniently administered. The primary factor in determining the amount of Formula (I) compound contained in oral doses is the required size of the delivery vehicle.

In still another aspect, the protective agent is a compound of formula (II):

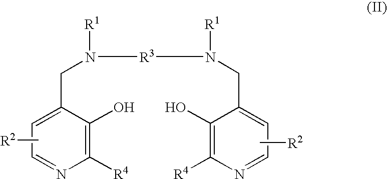

or a metal chelate thereof or salt of a metal chelate thereof, wherein in formula (II),

each R¹ independently represents hydrogen or —CH₂ COR⁵;

R⁵ represents hydroxy, optionally hydroxylated alkoxy, amino or alkylamido;

each R² independently represents a group XYR⁶;

X represents a bond, or a C₁₋₃ alkylene or oxoalkylene group optionally substituted by a group R⁷;

Y represents a bond, an oxygen atom or a group NR⁶;

R⁶ is a hydrogen atom, a group COOR⁸, an alkyl, alkenyl, cycloalkyl, aryl or aralkyl group optionally substituted by one or more groups selected from COOR.sup.8, CONR⁸₂, NR⁸₂, OR⁸, ═NR⁸, ═O, OP(O)(OR⁸)R⁷ and OSO₃ M;

R⁷ is hydroxy, an optionally hydroxylated, optionally alkoxylated alkyl or aminoalkyl group;

R⁸ is a hydrogen atom or an optionally hydroxylated, optionally alkoxylated alkyl group;

M is a hydrogen atom or one equivalent of a physiologically tolerable cation;

R³ represents a C₁₋₈ alkylene group, a 1,2-cycloalkylene group, or a 1,2-arylene group; and

each R⁴ independently represents hydrogen or C₁₋₃ alkyl.

Compounds of formula (II) and metal chelate thereof or salt of a metal chelate thereof, their preparations, administration, and uses for reducing cardiotoxicity of anthracyclines are disclosed in U.S. Pat. No. 6,147,094, the disclosure of which is incorporated herein by reference.

Compounds of formula (II) in which R³ is ethylene and R² has any of the identities listed above are particularly preferred.

Preferred metal chelates of the compounds for use in the method of the invention are those in which the metal ions are selected from the alkali and alkaline earth metals and from those metals having an atomic number from 22-31, 42, 44 and 58-70 and more particularly chelates having a Kₐ in the range from 10⁹ to 10²⁵, preferably 10¹⁰ to 10²⁴, more preferably 10¹¹ to 10²³. Particularly preferred chelates are those with metals other than iron which have a Kₐ value smaller, preferably by a factor of at least 10.sup.3, than the Kₐ value of the corresponding iron (Fe³⁺) chelate. Suitable ions include Na⁺, Mn²⁺, Cu⁺, Cu2+, Mg²⁺, Gd³⁺, Ca²⁺ and Zn²⁺ Mn²⁺ is especially preferred.

As chelates of aminopolycarboxylic acids, MnDTPA, MnEDTA, Mn DTPA.BMA and Mn EDTA.BMA are particularly preferred for use in accordance with the invention.

More particularly preferred for use in accordance with the invention is the compound N,N′-bis-(pyridoxal-5-phosphate)-ethylenediamine-N,N′-diacetic acid or N,N′-bis(3-hydroxy-2-methyl-5-phosphonomethyl-4-pyridyl-methyl)-ethylenedi amine-N,N′-diacetic acid (hereinafter referred to as DPDP) and the manganese chelate, Mn(DPDP).

If not all of the labile hydrogens of the chelates are substituted by the complexed metal ion, biotolerability and/or solubility of the chelate may be increased by substituting the remaining labile hydrogen atoms with physiologically biocompatible cations of inorganic and/or organic bases or amino acids. Examples of suitable inorganic cations include Li⁺, K⁺, Na⁺ and especially Ca²⁺. Suitable organic cations include ammonium, substituted ammonium, ethanolamine, diethanolamine, morpholine, glucamine, N,N,-dimethyl glucamine, lysine, arginine or omithine.

The compounds of formula (II) of the invention may be prepared by methods known in the art. Suitable methods for preparing the amino polycarboxylic acid based chelating agents are described in EP-A-299795, EP-A-71564, DE-A-3401052, EP-A-203962, EP-A-436579, EP-A-290047, and U.S. Pat. No. 6147094.

The compounds of formula (II) of the present invention may be formulated with conventional methods know in the art, such as that described in U.S. Pat. No. 6,147,094. For example, the compounds, optionally with the addition of pharmaceutically acceptable excipients, may be suspended or dissolved in an aqueous medium, with the resulting solution or suspension then being sterilized. Suitable additives include, for example, physiologically biocompatible buffers (e.g. tromethamine hydrochloride), additions (e.g. 0.01 to 10 mole percent) of chelants (such as, for example, DTPA and DTPA-bisamide) or calcium chelate complexes (e.g. calcium DTPA, CaNaDTPA-bisamide, or calcium salts), or, optionally, additions (e.g. 1 to 50 mole percent) of calcium or sodium salts (e.g. calcium chloride, calcium ascorbate, calcium gluconate or calcium lactate combined with metal chelate complexes of chelating agents according to the invention and the like). The compound may be in a conventional pharmaceutical administration form such as a tablet, capsule, powder, solution, suspension, dispersion, syrup, suppository, etc. However, solutions, suspensions and dispersions in physiologically acceptable carrier media, for example water for injections, will generally be preferred.

The preferred mode for administering the compounds of formula (II) in accordance with the invention is parenteral, e.g. intravenous administration. Parenterally administrable forms, e.g. intravenous solutions, should be sterile and free from physiologically unacceptable agents, and should have low osmolality to minimize irritation or other adverse effects upon administration, and thus the compositions should preferably be isotonic or slightly hypertonic. Suitable vehicles include aqueous vehicles customarily used for administering parenteral solutions such as Sodium Chloride Injection, Ringer's Injection, Dextrose Injection, Dextrose and Sodium Chloride Injection, Lactated Ringer's Injection and other solutions such as are described in Remington's Pharmaceutical Sciences, 15th ed., Easton: Mack Publishing Co., pp. 1405-1412 and 1461-1487 (1975) and The National Formulary XIV, 14th ed. Washington: American Pharmaceutical Association (1975). The solutions may contain preservatives, antimicrobial agents, buffers and antioxidants conventionally used for parenteral solutions, excipients and other additives which are compatible with the chelates and which will not interfere with the manufacture, storage or use of the products.

The compound of formula (II) in accordance with the invention may conveniently be administered in amounts of from 0.01 to 100 μmol of the compounds per kilogram of body weight, e.g. about 10 μmol per kg bodyweight. It may be administered simultaneously, separately or sequentially with the administration of the anthracycline.

In a further aspect the present invention provides a pharmaceutical packaging that comprises (a) a packaging material, (b) a pharmaceutical agent comprising an anthracycline, and (c) a written mater indicating that the pharmaceutical agent is for treating multiple sclerosis, wherein the pharmaceutical agent and the written matter are enclosed in the packaging material. The pharmaceutical packaging of the invention can be prepared by methods known in the art. Any packaging material suitable for packaging pharmaceuticals can be used in the invention.

### EXAMPLES

Without further elaboration, it is believed that one skilled in the art can, using the preceding description, practice the present invention to its fullest extent. The following detailed examples are provided to further illustrate the invention, and should not be construed as limitations of the preceding disclosure in any way whatsoever.

### Example 1

A female patient, 32 years of age, is diagnosed with progressive multiple sclerosis. Anthracycline therapy is initiated with doxorubicin by intravenous injection at a dose of 40 mg/m² on a 12-week cycle. Prior to the administration of the anthracycline, the patient is pretreated with 400 mg of dexrazoxane by intravenous injection about 30 minutes prior to administration of the doxorubicin. The patient is monitored for progress of treatment and for hematologic and non-hematologic toxicity throughout the course of treatment. The dose of doxorubicin is increased to 45 mg and the dose of dexrazoxane increased to 450 mg in the next cycle when the maximal hematologic and non-hematologic toxicity does not exceed grade 22 by NCI-CTC criteria.

### Example 2

A male patient, 25 years of age, is diagnosed with progressive multiple sclerosis. Anthracycline therapy with epirubicin 75 mg by intravenous injection is initiated on a 12-week cycle. Prior to the administration of the anthracycline, the patient is pretreated with dexrazoxane at 750 mg by intravenous injection. The patient is monitored for progress of treatment and hematologic and non-hematologic toxicity throughout the course of treatment. The dose is titillated to epirubicin 100 mg and dexrazoxane 1000 mg, in the second cycle of dose administration when the maximal hematologic and non-hematologic toxicity does not exceed grade 22 by NCI-CTC criteria. The patient's clinical condition deteriorates between weeks 9 and 12 during each of the first and second treatment cycles; accordingly, the treatment cycle is shortened to 8 weeks after the third dose.

### Example 3

A male patient, 30 years of age, is diagnosed with progressive multiple sclerosis. Anthracycline therapy with daunomycin at 40 mg is initiated with a 12-week cycle. The daunomycin is administered by a single intravenous injection. The patient is monitored for progress of treatment and for hematologic and non-hematologic toxicity throughout the course of treatment. The dose of daunomycin is increased to 60 mg starting in the second cycle of treatment when the maximal hematologic and non-hematologic toxicity in the patient does not exceed grade 22 by NCI-CTC criteria.

### Example 4

A male patient, 40 years of age, is diagnosed with progressive multiple sclerosis. Anthracycline therapy with idarubicin at 40 mg is initiated with a 12-week cycle. The idarubicin is administered by a single intravenous injection. The patient is monitored for progress of treatment and hematologic and non-hematologic toxicity throughout the course of treatment. The maximal hematologic and non-hematologic toxicity in the patient slightly exceeds grade 22 by NCI-CTC criteria following administration of each dose, and accordingly, the dose of idarubicin is not increased, but kept at 40 mg at subsequent cycles.

What is claimed is:

1. A method of treating multiple sclerosis in a patient in need of treatment comprising administering to the patient an therapeutically effective amount of an anthracycline or a pharmacologically acceptable salt thereof.

2. The method according to claim 1, wherein the anthracycline is administered intravenously.

3. The method according to claim 1, wherein the anthracycline is selected from the group consisting of doxorubicin, 13-deoxydoxorubicin, iodoxorubicin, daunorubicin, epirubicin, THP-adriamycin, idarubicin, menogaril, aclacinomycin A, zorubicin, pirarubicin, valirubicin, amrubicin, iodoxorubicin, nemorubicin, (4R)-1-(4-carboxy-1-oxobutyl)-4-hydroxy-L-prolyl-L-alanyl-L-seryl-(2R)-2-cyclohexylglycyl-L-glutaminyl-L-seryl-L-leucine, and 4′ deoxy-4′-iododoxorubicin.

4. The method according to claim 3, wherein the anthracycline is doxorubicin, derivative thereof, or a pharmacologically acceptable salt thereof.

5. The method according to claim 4, wherein doxorubicin or pharmacologically acceptable salt thereof is administered intravenously at an amount from about 10 to about 60 mg/m².

6. The method according to claim 4, wherein doxorubicin or pharmacologically acceptable salt thereof is administered intravenously at an amount from about 40 to about 45 mg/m².

7. The method according to claim 3, wherein the anthracycline is daunorubicin, derivative thereof, or a pharmacologically acceptable salt thereof.

8. The method according to claim 7, wherein daunorubicin or pharmacologically acceptable salt thereof is administered intravenously at an amount from about 30 to about 80 mg/m².

9. The method according to claim 7, wherein daunorubicin or pharmacologically acceptable salt thereof is administered intravenously at an amount from about 40 to about 60 mg/m².

10. The method according to claim 3, wherein the anthracycline is epirubicin or a pharmacologically acceptable salt thereof.

11. The method according to claim 10, wherein epirubicin or pharmacologically acceptable salt thereof is administered intravenously at an amount from about 30 to about 150 mg/m².

12. The method according to claim 10, wherein epirubicin or pharmacologically acceptable salt thereof is administered intravenously at an amount from about 75 to about 100 mg/m².

13. The method according to claim 3, wherein the anthracycline is idarubicin or a pharmacologically acceptable salt thereof.

14. The method according to claim 13, wherein idarubicin or pharmacologically acceptable salt thereof is administered intravenously or orally at an amount from about 12 to about 60 mg/m².

15. The method according to claim 13, wherein idarubicin or pharmacologically acceptable salt thereof is administered intravenously or orally at an amount from about 40 to about 60 mg/m².

16. The method according to claim 1, further comprising administering to the patient an effective amount of a protective agent.

17. The method according to claim 16 wherein said protective agent is bisdioxopiperazine or pharmaceutically acceptable salt thereof.

18. The method according to claim 17 wherein said bisdioxopiperazine is dexrazoxane.

19. The method according to claim 18, wherein dexrazoxane is administered intravenously.

20. The method according to claim 19, wherein dexrazoxane or pharmaceutically acceptable salt thereof is administered at an amount from about 100 to about 2500 mg/m².

21. The method according to claim 16 wherein said protective agent is a compound of formula (I), 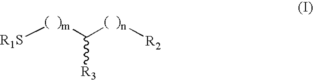
or pharmaceutically acceptable salt thereof, wherein in formula (I),
R1 is hydrogen, lower alkyl or 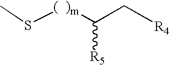
R₂ and R₄ are each individually SO₃⁻M⁺, PO₃²⁻M₂²⁺, or PO₂S²⁻M₂²⁺;
R₃ and R₅ are each individually hydrogen, hydroxy or sulfhydryl;
m and n are individually 0, 1, 2, 3 or 4, with the proviso that if m or n is 0, then R₃ is hydrogen; and
M is hydrogen or an alkali metal ion.

22. The method according to claim 21 wherein said compound of formula (I) is administered prior to administration of the anthracycline.

23. The method according to claim 21 wherein said compound of formula (I) compound is administered simultaneously with the anthracycline.

24. The method according to claim 21 wherein said formula (I) compound is administered to said patient intravenously.

25. The method according to claim 21 wherein said formula (I) compound is administered to said patient orally.

26. The method according to claim 16, wherein the anthracycline is selected from the group consisting of doxorubicin, 13-deoxydoxorubicin, iodoxorubicin, daunorubicin, epirubicin, THP-adriamycin, idarubicin, menogaril, aclacinomycin A, zorubicin, pirarubicin, valrubicin, amrubicin, iodoxorubicin, nemorubicin, (4R)-1-(4-carboxy-1-oxobutyl)-4-hydroxy-L-prolyl-L-alanyl-L-seryl-(2R)-2-cyclohexylglycyl-L-glutaminyl-L-seryl-L-leucine, 4′ deoxy-4′-iododoxorubicin, and a pharmacologically acceptable salt of any said anthracyclines.

27. The method according to claim 26 wherein said anthracycline is doxorubicin, derivative thereof, or a pharmacologically acceptable salt thereof.

28. The method according to claim 26 wherein said anthracycline is daunorubicin derivative thereof, or a pharmacologically acceptable salt thereof.

29. The method according to claim 26 wherein said anthracycline is epirubicin derivative thereof, or a pharmacologically acceptable salt thereof.

30. The method according to claim 26 wherein said anthracycline is idarubicin, derivative thereof, or a pharmacologically acceptable salt thereof.

31. The method according to claims 1 or 16 wherein the anthracycline is administered as an anthracycline prodrug.

32. The method according to claim 16 wherein said protective agent is a compound of formula (II), 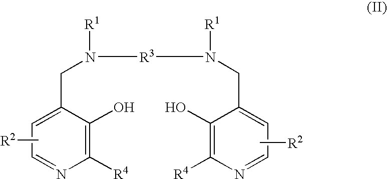
or a metal chelate thereof or salt of a metal chelate thereof, wherein in formula (II)
each R¹ independently represents hydrogen or —CH₂ COR⁵;
R⁵ represents hydroxy, optionally hydroxylated alkoxy, amino or alkylamido;
each R independently represents a group XYR⁶;
X represents a bond, or a C₁₋₃ alkylene or oxoalkylene group optionally substituted by a group R⁷;
Y represents a bond, an oxygen atom or a group NR⁶;
R⁶ is a hydrogen atom, a group COOR⁸, an alkyl, alkenyl, cycloalkyl, aryl or aralkyl group optionally substituted by one or more groups selected from COOR.sup.8, CONR⁸₂, NR⁸₂, OR⁸, ═NR⁸, ═O, OP(O)(OR⁸)R⁷ and OSO₃ M;
R⁷is hydroxy, an optionally hydroxylated, optionally alkoxylated alkyl or aminoalkyl group;
R⁸ is a hydrogen atom or an optionally hydroxylated, optionally alkoxylated alkyl group;
M is a hydrogen atom or one equivalent of a physiologically tolerable cation;
R³ represents a C₁₋₈ alkylene group, a 1,2-cycloalkylene group, or a 1,2-arylene group; and
each R⁴ independently represents hydrogen or C₁₋₃ alkyl.

33. The method according to claim 32 wherein said metal chelate comprises a metal ion selected from the group consisting of alkali and alkaline earth metals and metals having an atomic number of from 22-31, 42, 44 and 58-70.

34. The method according to claim 33 wherein said metal ion is selected from the group consisting of Na⁺, Mn²⁺, Cu⁺, CU²⁺, Mg²⁺, Gd³⁺, Ca²⁺ and Zn²⁺.

35. The method according to claim 32 wherein said chelate is manganese chelate and has a Kₐ in the range of from 10⁹ to 10²⁵.

36. The method according to claim 35 wherein said manganese chelate has a Kₐ in the range of from 10¹² to 10²².

37. The method as claimed in claim 32 wherein said chelate is manganese chelate and has a Kₐ value smaller by a factor of at least 10³ than the Kₐ value of the corresponding iron (Fe³⁺) chelate.

38. A pharmaceutical packaging comprising: (a) a packaging material, (b) a pharmaceutical agent comprising an anthracycline, and (c) a written mater indicating the pharmaceutical agent is for treating multiple sclerosis, wherein the pharmaceutical agent and the written matter are enclosed in the packaging material.
